(19) United States
(12) Patent Application Publication
(10) Pub. No.: US 2014/0121721 A1
(43) Pub. Date: May 1, 2014

(54) APPARATUS AND METHOD FOR PLACEMENT OF LEAD FOR CARDIAC RESYNCHRONIZATION THERAPY

(71) Applicant: MEDTRONICS, INC., Minneapolis, MN (US)

(72) Inventors: Raja N. Ghanem, Edina, MN (US); Manfred Justen, Lino Lakes, MN (US)

(73) Assignee: MEDTRONIC, INC.

(21) Appl. No.: 13/663,979

(22) Filed: Oct. 30, 2012

(51) Int. Cl. A61N 1/37

(52) U.S. Cl. CPC A61N 1/371
USPC 607/28

(57) ABSTRACT

An apparatus and method for placement of a lead for cardiac resynchronization therapy in a cardiovascular system of patient. A conductive tool is advanced along at least one branch of the cardiovascular system of the patient. Electrogram data of the cardiovascular system at each location of the conductive tool along the cardiovascular system using the conductive tool is obtained while the conductive tool is advanced. The electrogram data is analyzed to determine a morphological condition of tissue of the patient surrounding the location.

### FIELD

The present invention relates generally to cardiac resynchronization therapy and, more particularly, to apparatus and methods for placement of leads for cardiac resynchronization therapy.

### BACKGROUND

Cardiac resynchronization therapy (“CRT”), also sometimes known as biventricular pacing, is a well known technique utilized in some patients having been diagnosed with congestive heart failure. CRT uses an implantable medical device, sometimes referred to as a pacemaker to re-coordinate the action of the right and left ventricles in patients with heart failure. In some patients with heart failure, an abnormality in the heart's electrical conducting system may cause a patient's two ventricles to beat in an asynchronous fashion. That is, instead of beating simultaneously, the two ventricles beat slightly out of phase. This asynchrony may reduce the efficiency of the ventricles in patients with heart failure, whose hearts are already damaged. CRT re-coordinates the beating of the two ventricles by pacing both ventricles simultaneously. This differs from typical pacemakers, which pace only the right ventricle. When the work of the two ventricles is coordinated, the heart's efficiency increases and the amount of work it takes for the heart to pump blood is reduced.

Studies with CRT have demonstrated its ability to improve the symptoms, the exercise capacity, and the feeling of well-being of many patients with moderate to severe heart failure. Studies have also shown that CRT may improve both the anatomy and function of the heart—tending to reduce the size of the dilated left ventricle, and therefore improving the left ventricular ejection fraction. Perhaps most importantly, CRT may improve the survival of patients with heart failure.

An implantable medical device used for CRT sends small, undetectable electrical impulses to both lower chambers of the heart to help them beat together in a more synchronized pattern. This improves the heart's ability to pump blood and oxygen to the body. Insulated wires, called leads, are implanted for two purposes: to carry information signals from your heart to the heart device and to carry electrical impulses to your heart.

Proper or optimal operation of an implantable medical device used for CRT relies on the proper placement of such leads in and/or around cardiac tissue. Since implantable medical devices used for CRT are typically implanted in patients that already have heart disease, a portion of the tissue of the patient's heart may be damaged, i.e., may be slow conducting, including scar tissue, or may be ischemic tissue. Such slow conducting or ischemic tissue may not transmit electrical signals either from or to the heart preventing an implantable medical device for proper or optimal operation.

### SUMMARY

In order to improve cardiac resynchronization therapy response, it may be important to ensure that the leads, especially the left ventricular lead, is placed in viable tissue. In other words, it may be important that the leads are not placed in such slow conducting scar or ischemic tissue. The placement of a lead or leads for CRT may be improved by collecting electrogram signals from a conductor, a conductive tool, which may be a left ventricular lead, while the conductive tool is being advanced through, or moved down, a branch of the coronary system. Collected electrogram signals from the left ventricular lead, catheter or guidewire, while these tools are moving through the coronary system, may be analyzed using waveform analysis algorithms, such as Wavelet, to identify ischemic tissue or scar tissue and avoid left ventricular lead placements in such tissue. Or conversely, such analysis conducted while the conductive tool is being advanced through the coronary system may be used to ensure that the location selected for placement of the lead is viable tissue, i.e., that scar tissue or ischemic tissue has been avoided. If such scar tissue or ischemic tissue has been avoided, then advancement of the conductive tool may halt with the lead placement location selected. If however, such scar tissue or ischemic tissue has not been avoided, then advancement, or withdrawal, of the conductive tool may proceed further until viable tissue has been located.

In an embodiment, a device-implemented method is for placement of a lead for cardiac resynchronization therapy in a cardiovascular system of patient. A conductive tool is advanced along at least one branch of the cardiovascular system of the patient. Electrogram data of the cardiovascular system at each location of the conductive tool along the cardiovascular system using the conductive tool is obtained while the conductive tool is advanced. The electrogram data is analyzed to determine a morphological condition of tissue of the patient surrounding the location.

In an embodiment, an apparatus determines appropriate placement of a lead for cardiac resynchronization therapy in a cardiovascular system of patient. A conductive tool is configured to be advanced along at least one branch of the cardiovascular system of the patient. A generator is operatively coupled to the conductive tool, the generator being configured to obtain electrogram data of the cardiovascular system at each location of the conductive tool along the cardiovascular system using the conductive tool. An analyzer, operatively coupled to the electrogram data, is configured to determine a morphological condition of tissue of the patient surrounding the location.

In an embodiment, the conductive tool is continuously advanced along a portion of the cardiovascular system of the patient.

In an embodiment, a recommendation on placement of the lead in the cardiovascular system of the patient based on the morphological condition of the tissue to avoid slow conducting tissue is provided.

In an embodiment, the conductive tool is electrically isolated from the patient along the conductive tool from a proximal end to a distal end.

In an embodiment, a bipolar measurement is made utilizing the distal end of the conductive tool and the proximal end of said conductive tool.

In an embodiment, a unipolar measurement is made utilizing the distal end of the conductive tool and a remote reference.

In an embodiment, the conductive tool is a test electrode.

In an embodiment, the conductive tool is an electrode for cardiac resynchronization therapy.

In an embodiment, advancement of the conductive tool is halted when the distal end of the lead arrives in a vicinity of intended stimulation for the cardiac resynchronization therapy and the morphological condition of the tissue of the patient of the location of the distal end of the lead is indicative of the tissue of the patient being suitable for the cardiac resynchronization therapy utilizing, at least in part, information derived from the analyzing step.

In an embodiment, advancing of the conductive tool is adjusted while the distal end of the lead is in a vicinity of intended stimulation for the cardiac resynchronization therapy based, at least in part, on the morphological condition of the tissue of the patient of the location of the distal end of the lead is indicative of the tissue of the patient being suitable for the cardiac resynchronization therapy utilizing, at least in part, information derived from analyzing electrogram data.

### FIGURES

FIG. 1 is a block diagram illustrating an embodiment in which a conductive tool is advanced in a branch of the vascular system of a patient represented by heart;

FIG. 2 shows unipolar electrograms recorded over an infarct region in a canine experiment;

FIG. 3 is an example of epicardial potentials overlaying an endocardial scar;

FIG. 4 is a mathematical picture of the signal representing an electrogram signal;

FIG. 5 illustrates a “percentage match” indicative how similar an electrogram is to a template;

FIG. 6 illustrates a Medtronic Analyzer and Programmer device;

FIG. 7 is an illustration of the use of fluoro to identify the conductive tool and navigate it through a 3D model; and

FIG. 8 is a flow chart of an embodiment.

### DESCRIPTION

The position of the left ventricular lead may play an important role for CRT response. Several studies have shown that the number of patients that may benefit from CRT may increase when the left ventricular lead is placed in a location, at a site, of latest mechanical activation or latest electrical activation. A number of prior art techniques are well known that describe methods or techniques to determine the mechanical and electrical activation of a particular location of the heart.

While a usually physically optimal location for a lead, e.g., a left ventricular lead, can be determined and such lead can be placed in close proximity to that location, there is little certainty that the location selected, the location where the lead has been placed contains viable tissue or whether tissue surrounding the location is viable. Stated conversely, the lead may have been placed in a location which does not contain or is not surrounded by viable tissue. As noted above, the tissue may be slow conducting scar tissue or ischemic tissue. Known techniques for determining the mechanical and electrical activation of the selected location may determine whether the location where the lead has been placed is viable, such techniques are not helpful in determining where to place and how to place the lead.

The placement of a lead or leads for CRT is improved by collecting electrogram signals from a conductor, a conductive tool, which may be a left ventricular lead, while the conductive tool is being advanced through, or moved down, a branch of the coronary system. Collected electrogram signals from the left ventricular lead, catheter or guidewire, while these tools are moving through the coronary system, are analyzed using waveform analysis algorithms, such as Wavelet, to identify ischemic tissue or scar tissue and avoid left ventricular lead placements in such tissue. Or conversely, such analysis is conducted while the conductive tool is being advanced through the coronary system to ensure that the location selected for placement of the lead is viable tissue, i.e., that scar tissue or ischemic tissue has been avoided. If such scar tissue or ischemic tissue has been avoided, then advancement of the conductive tool is halted with the lead placement location selected. If however, such scar tissue or ischemic tissue has not been avoided, then advancement, or withdrawal, of the conductive tool may proceed further or be otherwise adjusted until viable tissue has been located.

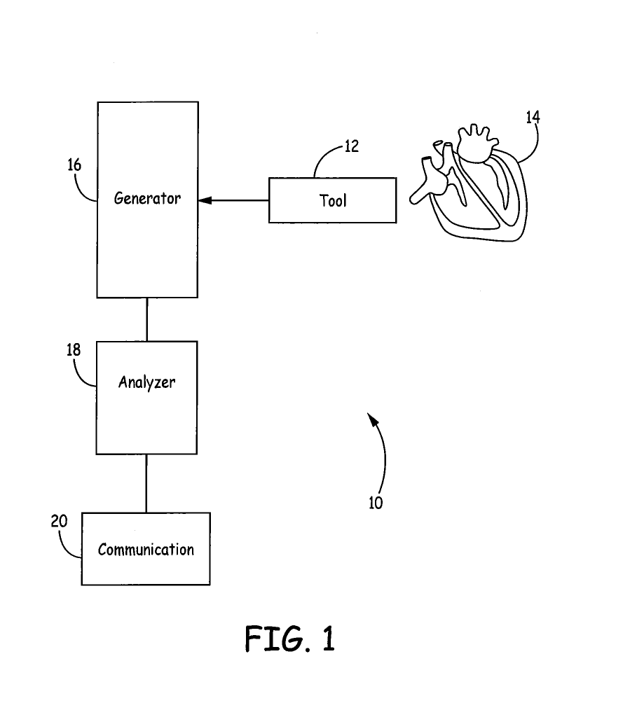

FIG. 1 is a block diagram illustrating an embodiment 10 in which a conductive tool 12 is advanced in a branch of the vascular system of a patient represented by heart 14. It is to be recognized that heart 14 serves only as a representation of the vascular system of the patient. Conductive tool 12 may be advanced not only in the heart but also in any of the coronary system of the heart and vascular structure leading to the heart. Conductive tool may be a lead, such as a left ventricle lead, or may be a catheter or guidewire. As an example, left ventricle lead may be advanced down a branch of the vascular system using a catheter. Either the catheter itself or a separate guidewire may be used as conductive tool 12 instead of or in addition to the lead.

Generator 16 operates to obtain electrogram data utilizing conductive lead while conductive lead is being advanced through the coronary system of the patient. As the electrogram data is obtained, analyzer 18 receives the electrogram data and the signals analyzed for morphological changes that would indicate slow conducting tissue, such as scar tissue or ischemic tissue, including an analysis of timing information that indicates normal myocardial activation. Thus, analyzer 18 can determine whether tissue at a location of conductive tool 12 or in the vicinity of conductive tool 12, as conductive tool 12 is advanced through the vascular system, is viable or not, i.e., whether the such tissue indicates normal myocardial activation or is slow conducting, e.g., scar tissue or ischemic tissue. Communication module 20, operatively coupled to analyzer 18, may then indicate to a user the nature of tissue of at or in the vicinity of conductive tool 12. A user may then adjust the position, location, of conductive tool 12 to better suit placement of a lead, e.g., the left ventricular lead. As an example, as conductive tool 12 approaches an area or vicinity intended for placement of the lead, communication module 20 will indicate the nature of the surrounding tissue. If communication module 20 indicates that the surrounding tissue is viable, then the lead may be placed at or near such location, for example by halting the advancement of conductive tool 12. However, if communication module 20 indicates that the surrounding tissue is slow conducting, then the location for the lead may be adjusted to find a better location for the lead. Generally, slow conducting tissue is tissue that is evidenced in fractionated electrograms with high frequency deflections. Fractionations are pertinent when EGMs are recording using bipolar electrodes. However, with unipolar signals, i.e., one electrode on the conductive tool and the other is a reference electrode far from the heart, then slow conduction or areas of infarction are depicted as negative going deflections, e.g., Q-waves.

Conductive tool 12 may continue to be advanced in the coronary system to attempt to find a better location with more/better viable tissue or the location of conductive tool 12 may be at least partially withdrawn also to attempt to find a better location with more/better viable tissue. If the lead is separate from conductive tool 12 and conductive tool 12 has already passed a location with viable tissue and a later location of conductive tool 12 indicates a lack of viable tissue, then a location for placement of the lead with viable tissue is already know and the lead may be placed in such location without necessarily repositioning conductive tool 12. Thus, it can be seen that communication module 20 may be used to either indicate that tissue surrounding a particular location of conductive tool 12 contains either viable tissue or lack viable tissue and either indication can be useful in determining a placement location for the lead which has or is surrounded by viable tissue.

Analyzer 18 considers waveforms from electrogram data form generator 16 of sensed events from the left ventricular lead, a catheter or guidewire to determine whether the potential target site is surrounded by viable tissue. The wavefoms of sensed events are going to change when the electrodes moves into an infarct region. Waveform analysis algorithms are able to identify changes of morphologies relative to a template or relative to each other.

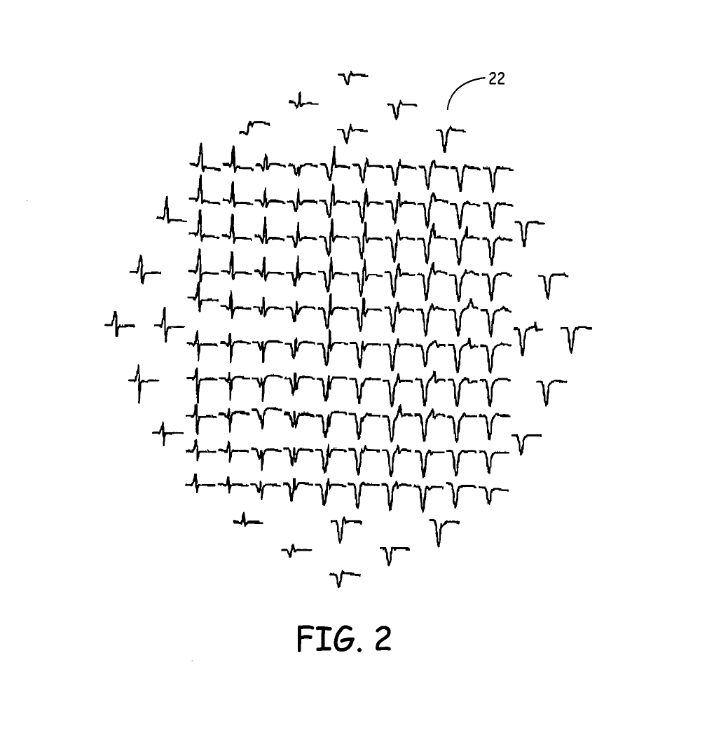

FIG. 2 shows unipolar electrograms 22 recorded over the infarct region in a canine experiment consisting of a set of 128 electrograms obtained over the infarct in the canine model. The electrode diameter is six (6) centimeters. The recordings are unipolar, with a right leg reference, obtained during sinus rhythm. Note that the biphasic QRS waveform on the left changes to morphology with a prominent Q-wave as the activation spreads over the infarct. Note also the emergence of the small late potentials following the QRS over the infarct. It is possible that electrogram based features could be developed to detect the Q-wave and late potential formation. Alternatively, a wavelet based approach can be used to compare the QRS morphology to a stored template that identifies the infarct (e.g. a Q-wave followed by a sharp R-wave).

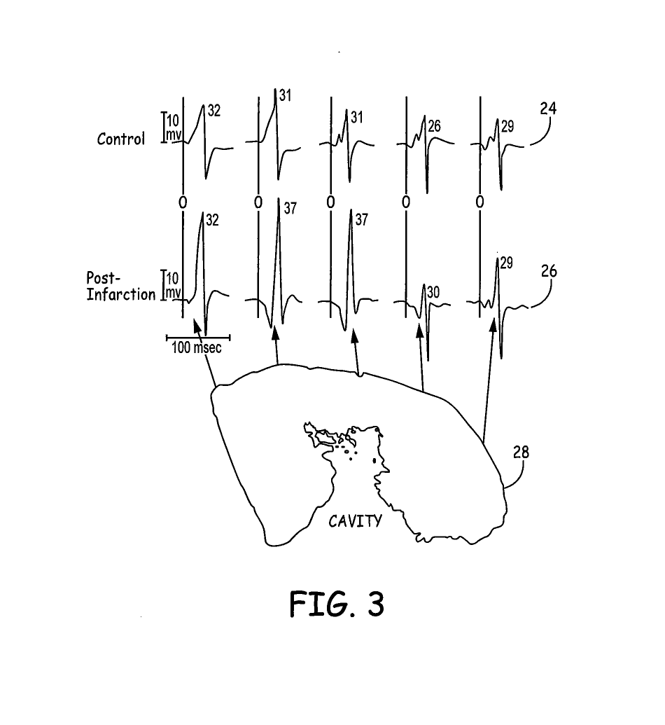

An additional example is shown in FIG. 3 which is an example of epicardial potentials overlaying an endocardial scar. Here the top panel 24 shows the normal biphasic QRS morphology over the epicardium. However, the morphology of the epicardial electrogram (middle panel 26) changes as conductive tool 12 is advanced to an infracted endocardial scar region 28. Again, a prominent Q-wave followed by a sharp tall R-wave signifying delayed activation is seen. It is feasible to construct a pattern of electrogram morphologies that will indicate scar. Such electrogram morphology can then be used as a template for the wavelet algorithm.

As an example, a Wavelet algorithm in our ICDs to analyze electrogram waveforms during detected ventricular arrhythmias and compare them to a reference waveform from an intrinsic sinus rhythm. If the waveforms are similar the rhythm is classified as sinus rhythm and therapy is withheld.

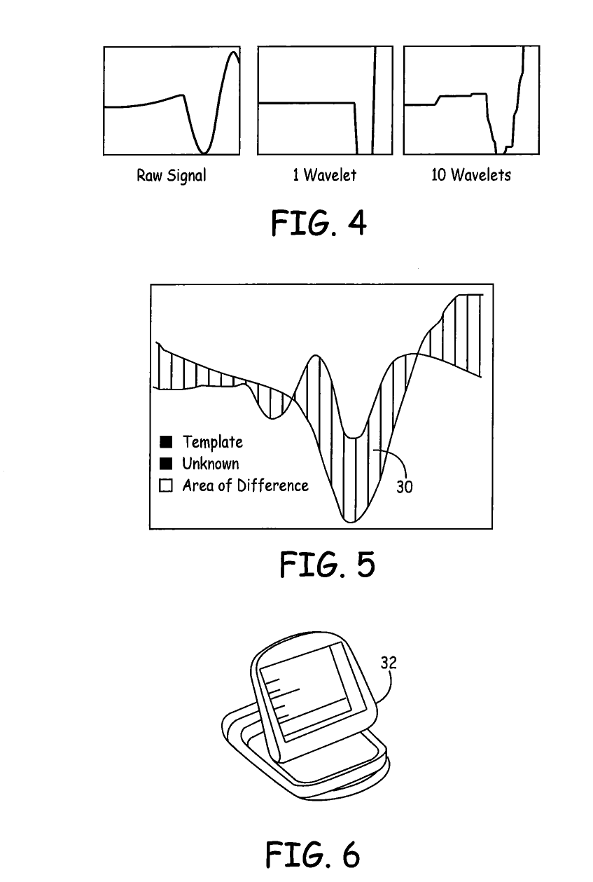

Wavelet breaks down the electrogram signal into a mathematical expression, using a function called Haar wavelet (lower case “w”) transform. That expression represents the signal as a single square waveform. Then, other parts of the waveform are represented by additional mathematical expressions. The more wavelets are applied, the better mathematical picture of the signal (FIG. 4) representing an electrogram signal using ten wavelet expressions.

By converting the signals to mathematical expressions, and using those expressions rather than the raw signal data, the ICD is able to more efficiently process the data needed, minimize battery drain, and perform a high resolution template matching procedure on a beat-to-beat basis during the procedure.

Generator 16 provides the electrogram waveform from the left ventricular lead, catheter or guidewire. Wavelet works by aligning and comparing the template and the unknown waveform(s), and determines the area of difference between the two signals. For each beat, the device returns a “percentage match,” indicating how similar the beat is to the template. Percentage match is calculated as 1-(area of difference). See area of difference 30 between the reference signal (template) and the unknown signal (FIG. 5).

The waveforms are collected through generator 16. The Waveform analysis is performed by analyzer 18 through a separate application that runs independent from generator 16 and continuously collects the electrogram signals from the left ventricular lead (e.g. a bipolar lead or the RV tip to left ventricular tip signal) or an electrical active catheter or guidewire. The template would be chosen based on the user's discretion.

There are three methods. First, the user defines a patient specific template. The user can decide to define one template at the beginning of the mapping procedure once the Coronary Sinus has been cannulated and compare each sensed event to this template while the left ventricular lead, catheter, or guidewire is navigated through the coronary system. Second, the system would compare waveforms relative to each other, which means that each new waveform will be compared to the previous waveform (template) while the left ventricular lead, catheter, or guidewire is navigated through the coronary system. Third, the system would use pre-defined templates that indicate scar and compares each sensed event to this template while the left ventricular lead, catheter, or guidewire is navigated through the coronary system.

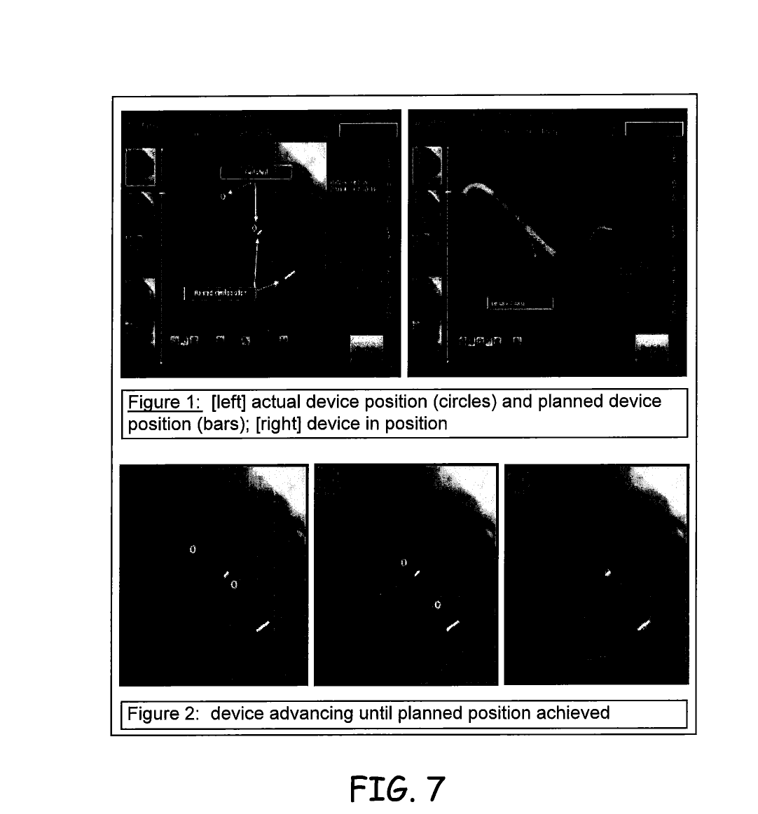

As an example, FIG. 6 illustrates a Medtronic Analyzer and Programmer device 32, Medtronic, Inc., Minneapolis, Minn., which can be used to assist with generator 16, analyzer 18 and communication module 20. Device 32 displays the electrogram and electrical signals from up to two electrodes, calculates signal strengths from each electrode and exports video signals to an external monitor.

The catheter and guidewire are used to support the positioning of the left ventricular lead. Users can get the guidewire into basically any branch of the coronary system. Once in place the left ventricular lead is moved over the wire into its location. A conventional guidewire and catheter may be modified in at least one of the following ways.

First, the guidewire is isolated except at the proximal end and distal end. The distal end (tip) continuously collects electrical information and the proximal end is connected to a Medtronic Analyzer or Programmer to process and display the signals. The signal is collected between the guidewire and the right ventricular lead (Nearfield EGM).

Second, a bipolar guidewire is utilized that contains two electrical active electrodes at its tip and allows sampling true bipolar signals. In this case an extra cable is added to the first option above.

Third, one (unipolar) or two (true bipolar) active electrodes is/are added to the catheter.

The locations of the different morphologies or areas with similar morphologies are displayed on a 3D venogram using a color code scheme. The anatomical information for the 3D venogram would be obtained through the Medtronic CardioGuide™ Implant System, Medtronic, Inc., Minneapolis, Minn., which creates a 3D model of the coronary system from C-Arm projection angles from a fluoroscopic system inside the operating room. The software would then use fluoroscopy to identify the real-time location of conductive tool 12, i.e., the guidewire or lead, and display the position of the 3D model (see FIG. 7). The areas with signal that do not match the template can be identified on the 3D model and marked as “No-go” zones.

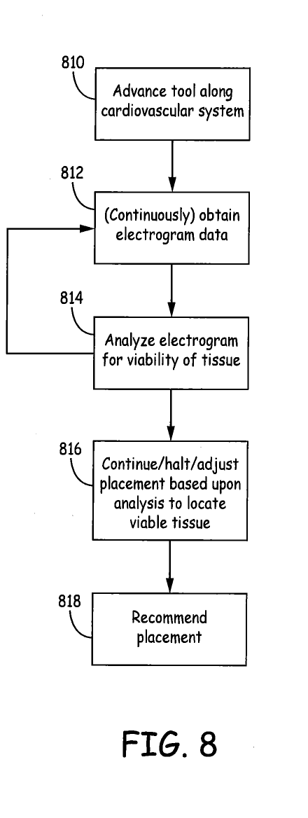

FIG. 8 is a flow chart illustrating an embodiment. Conductive tool 12 is advanced 810 along at least one branch of the cardiovascular/coronary system of the patient. During the such advancement, electrogram data is obtained 812, preferably continuously, during the advancement at each location, or a plurality of locations, of the conductive tool 12 along the cardiovascular system. Electrogram data is analyzed 814, as discussed above, to determine a morphological condition of tissue at or surrounding each such location or each of the plurality of locations. The morphological condition determines the viability of the tissue, e.g., the presence or absence of slow conducting tissue such as scar tissue or ischemic tissue. Advancement of conductive tool 12 may be adjusted 816, e.g., halted or continued, or the placement location for the lead may otherwise may be recommended 818 to be adjusted to locate a placement for the lead at a location having or surrounded by viable tissue.

What is claimed is:

1. A device-implemented method for placement of a lead for cardiac resynchronization therapy in a cardiovascular system of patient, comprising the steps of:
advancing a conductive tool along at least one branch of said cardiovascular system of said patient;
during said advancing step, obtaining electrogram data of said cardiovascular system at each location of said conductive tool along said cardiovascular system using said conductive tool; and
analyzing said electrogram data to determine a morphological condition of tissue of said patient surrounding said location.

2. The method of claim 1 wherein said obtaining step is performed continuously as said conductive tool is advanced along a portion of said cardiovascular system of said patient.

3. The method of claim 1 further comprising the step of providing a recommendation on placement of said lead in said cardiovascular system of said patient based on said morphological condition of said tissue to avoid slow conducting tissue.

4. The method of claim 1 wherein said conductive tool is electrically isolated from said patient along said conductive tool from a proximal end to a distal end.

5. The method of claim 4 wherein said obtaining step is a bipolar measurement utilizing said distal end of said conductive tool and said proximal end of said conductive tool.

6. The method of claim 4 wherein said obtaining step is a unipolar measurement utilizing said distal end of said conductive tool and a remote reference.

7. The method of claim 4 wherein said conductive tool comprises a test electrode.

8. The method of claim 4 wherein said conductive tool comprises an electrode for cardiac resynchronization therapy.

9. The method of claim 8 wherein said obtaining step is performed continuously as said lead is advanced along a portion of said cardiovascular system of said patient.

10. The method of claim 9 wherein said advancing step is halted when said distal end of said lead arrives in a vicinity of intended stimulation for said cardiac resynchronization therapy and said morphological condition of said tissue of said patient of said location of said distal end of said lead is indicative of said tissue of said patient being suitable for said cardiac resynchronization therapy utilizing, at least in part, information derived from said analyzing step.

11. The method of claim 9 wherein said advancing step is adjusted while said distal end of said lead is in a vicinity of intended stimulation for said cardiac resynchronization therapy based, at least in part, on said morphological condition of said tissue of said patient of said location of said distal end of said lead is indicative of said tissue of said patient being suitable for said cardiac resynchronization therapy utilizing, at least in part, information derived from said analyzing step.

12. An apparatus determining appropriate placement of a lead for cardiac resynchronization therapy in a cardiovascular system of patient, comprising:
a conductive tool configured to be advanced along at least one branch of said cardiovascular system of said patient;
a generator operatively coupled to said conductive tool, said generator being configured to obtain electrogram data of said cardiovascular system at each location of said conductive tool along said cardiovascular system using said conductive tool; and
an analyzer, operatively coupled to said electrogram data, said analyzer configured to determine a morphological condition of tissue of said patient surrounding said location.

13. The apparatus of claim 12 wherein said generator is configured to continuously obtain said electrogram data as said conductive tool is advanced along a portion of said cardiovascular system of said patient.

14. The apparatus of claim 12 further comprising an output configured to provide a recommendation on placement of said lead in said cardiovascular system of said patient based on said morphological condition of said tissue to avoid slow conducting tissue.

15. The apparatus of claim 12 wherein said conductive tool is electrically isolating from a proximal end to a distal end.

16. The apparatus of claim 15 wherein said electrogram data is obtained with a bipolar measurement utilizing said distal end of said conductive tool and a proximal end of said conductive tool.

17. The apparatus of claim 15 wherein said electrogram data is obtained with a unipolar measurement utilizing said distal end of said conductive tool.

18. The apparatus of claim 15 wherein said conductive tool comprises a test electrode.

19. The apparatus of claim 15 wherein said conductive tool comprises an electrode for cardiac resynchronization therapy.

20. The apparatus of claim 19 wherein said generator is configured to continuously obtain said electrogram data as said conductive tool is advanced along a portion of said cardiovascular system of said patient.

21. The apparatus of claim 20 wherein advancement of said lead is halted when said distal end of said lead arrives in a vicinity of intended stimulation for said cardiac resynchronization therapy and said morphological condition of said tissue of said patient of said location of said distal end of said lead is indicative of said tissue of said patient being suitable for said cardiac resynchronization therapy.

22. The apparatus of claim 20 wherein advancement of said lead is adjusted while said distal end of said lead is in a vicinity of intended stimulation for said cardiac resynchronization therapy based, at least in part, said morphological condition of said tissue of said patient of said location of said distal end of said lead is indicative of said tissue of said patient being suitable for said cardiac resynchronization therapy.
